(19) United States
(12) Patent Application Publication
(10) Pub. No.: US 2009/0274740 A1
(43) Pub. Date: Nov. 5, 2009

(54) DRUG-LOADED MEDICAL DEVICES AND METHODS FOR MANUFACTURING DRUG-LOADED MEDICAL DEVICES

(75) Inventors: Michael Kuehling, Munich (DE); Yixin XU, Newton, MA (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 12/433,341

(22) Filed: Apr. 30, 2009

Related U.S. Application Data
(60) Provisional application No. 61/049,562, filed on May 1, 2008.

(51) Int. Cl. A61K 9/00; B05D 1/00; A61P 43/00

(52) U.S. Cl. 424/423; 427/2.25; 424/400

(57) ABSTRACT

Drug-loaded medical devices such as stents and methods for manufacturing them are provided. In certain embodiments, the therapeutic agent is loaded only in or on areas of the medical device that do not undergo substantial deformation during expansion. The medical device may be provided with reservoirs in or on the first portions of the medical device that do not undergo substantial deformation during expansion, and the therapeutic agent may be loaded in these reservoirs. The reservoirs may be covered with a porous coating which may be a non-polymeric coating. In other embodiments, a non-polymeric coating is provided in a discontinuous manner in or on the medical device. A method includes the steps of providing a transfer member that includes a translucent substrate and a layer of transfer material such as a film or foil; attaching reservoirs to the transfer material; positioning the transfer member with the reservoirs adjacent a medical device, such as a stent, with the reservoirs facing the medical device; and directing a laser beam at the transfer member such that the laser beam passes through the translucent substrate to vaporize a portion of the transfer material, creating a pocket of trapped gas between the translucent substrate and reservoir, thereby forcing the reservoir from the transfer member to the medical device. The reservoirs may be formed of a non-polymeric material.

### CROSS REFERENCE TO RELATED APPLICATION

The present application claims priority to U.S. provisional application Ser. No. 61/049,562 filed May 1, 2008, the disclosure of which is incorporated herein by reference in its entirety.

### TECHNICAL FIELD

The present application generally relates to drug-loaded medical devices, such as stents, and to methods for manufacturing such drug-loaded medical devices.

### BACKGROUND

The positioning and deployment of implantable medical devices within a target site of a patient are common, often repeated, procedures of contemporary medicine. These devices, which may be implantable stents, chronic rhythm management leads, neuromodulation devices, implants, grafts, defibrillators, filters, and catheters, as well as other devices, may be deployed for short or sustained periods of time and may be used for many medicinal purposes. These can include the reinforcement of recently re-enlarged lumens, the replacement of ruptured vessels, and the treatment of disease, such as vascular disease, through the delivery of therapeutic agent.

Coatings may be applied to the surfaces of implantable medical devices to transport therapeutic agent to a target site and to release it at the target site. Coatings may also be provided for other purposes, such as radiopacity or biocompatibility. Many coating methods have been proposed, including dip coating, spray coating, etc. In certain instances, it is desired to apply precise amounts of coating to specific areas of the device. For such applications, coating by a nozzle, for example applying ink-jet technology, has been proposed.

### BRIEF DESCRIPTION

Certain embodiments of the present invention are directed to drug-loaded medical devices wherein the drug or therapeutic agent is loaded only in or on areas of the medical device that do not undergo substantial deformation during expansion. In these embodiments, the medical device is an expandable medical device, such as a stent, that undergoes expansion during deployment. The medical device has one or more first portion(s) that do not undergo substantial deformation during expansion of the medical device and one or more second portion(s) that undergo substantial deformation during expansion of the medical device. The therapeutic agent is loaded only in or on the first portion(s) of the medical device. The medical device may be provided with one or more reservoirs in or on the first portion(s) of the medical device, and the therapeutic agent may be loaded in these reservoirs. The reservoirs may be covered with a porous coating which may be a non-polymeric coating.

Other embodiments of the present invention are directed to methods for manufacturing drug-loaded medical devices wherein the therapeutic agent is loaded only in or on areas of the medical device that do not undergo substantial deformation during expansion. In these embodiments, the method includes providing an expandable medical device, such as a stent, that undergoes expansion during deployment. The medical device has one or more first portion(s) that do not undergo substantial deformation during expansion of the medical device and one or more second portion(s) that undergo substantial deformation during expansion of the medical device. The method includes loading the therapeutic agent only in or on the first portion(s) of the medical device. The method may include providing one or more reservoirs in or on the first portion(s) of the medical device, and loading the therapeutic agent in these reservoirs. The method may include covering the reservoirs with a porous coating which may be a non-polymeric coating.

Other embodiments of the present invention are directed to methods for manufacturing drug-loaded medical devices wherein the therapeutic agent is loaded in a discontinuous manner in or on the medical device. This method includes the steps of providing a transfer member that includes a translucent substrate and a layer of transfer material such as a film or foil; attaching one or more reservoirs to the transfer material; positioning the transfer member with the reservoirs adjacent a medical device, such as a stent, with the reservoirs facing the medical device; and directing a laser beam at the transfer member such that the laser beam passes through the translucent substrate to vaporize a portion of the transfer material to create a pocket of trapped gas between the translucent substrate and reservoir to force the reservoir from the transfer member to the medical device. The reservoirs are loaded with therapeutic agent. The loading of the therapeutic agent may take place prior to or after attaching the reservoirs to the transfer member, or prior to or after transferring the reservoirs from the transfer member to the medical device. The reservoirs may be formed of a non-polymeric material.

The invention may be embodied by numerous other devices and methods. The description provided herein, when taken in conjunction with the annexed drawings, discloses examples of the invention. Other embodiments, which incorporate some or all steps as taught herein, are also possible.

### BRIEF DESCRIPTION OF THE DRAWINGS

Referring to the drawings, which form a part of this disclosure:

FIG. 1 shows a stent that may be coated in accordance with certain embodiments of the present invention;

FIG. 2 shows an enlarged view of a stent strut of the stent of FIG. 1;

FIGS. 3a-c show side views of a straight section of the stent strut of FIG. 2 during various method steps that may be employed in accordance with embodiments of the present invention; and

FIGS. 4a-b show a stent being coated in accordance with another embodiment of the present invention.

### DETAILED DESCRIPTION

The present invention generally relates to methods for coating medical devices. Medical devices which may be coated in accordance with embodiments of the present invention include, but are not limited to, implantable stents, chronic rhythm management leads, neuromodulation devices, implants, grafts, defibrillators, filters, and catheters. The medical devices themselves may be self-expanding, mechanically expandable, such as by balloon or other expander, or hybrid implants which may have both self-expanding and mechanically expandable characteristics. The medical devices may be made in a wide variety of designs and configurations. Additionally, the medical devices may be fabricated from various materials including conductive materials, such as conductive ceramic, bio-ceramic, polymeric, metallic, and other bio-compatible materials.

The coatings discussed herein may also be comprised of ceramic, bio-ceramic, polymeric, metallic, and other bio-compatible materials. These coating may be polymer-free and, thus, may eliminate any potentially inflammatory reactions associated with the use of polymers on medical devices.

Polymer-free coatings, for example, polymer-free ceramic coatings, may not be as robust as polymeric coatings. As a result, these polymer-free coatings may be damaged during deformation.

For instance, in the case of a stent, the stent coating may be as thin as 200-500 nanometers. Thus, the coating can be fragile and face challenges when the stent is expanded during deployment. For example, the coating may flake off, or fracture, during expansion of the stent. Accordingly, there is a need for coatings which can survive high stress and strain caused by the expansion of the stent during deployment.

To address the drawbacks associated with polymer-free coatings, certain embodiments of the present invention provide drug-loaded stents wherein the polymer-free coating is discontinuous and not subject to significant stress/strain during stent expansion. This allows the stent to expand without causing damage to the coating.

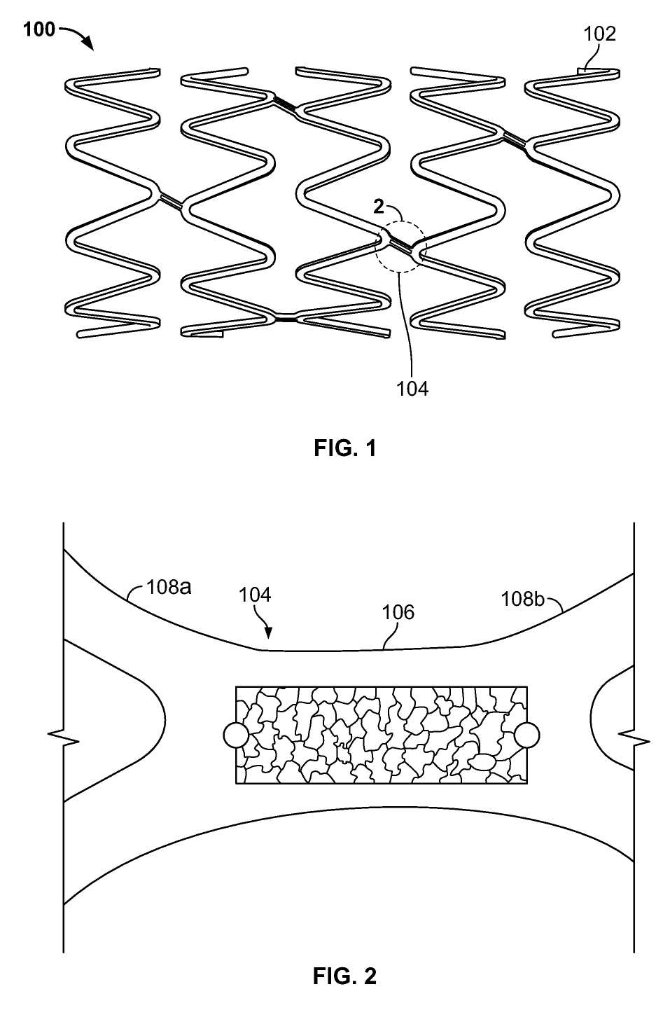

Referring initially to FIG. 1, a stent 100 is shown having a lattice portion 102 comprised of a plurality of stent struts 104.

FIG. 2 shows an enlarged view of the stent strut 104 of FIG. 1. As seen in the example of FIG. 2, each stent strut 104 is comprised of a straight section 106, and located on either side of the straight section, are Y-shaped sections 108a, b. In other embodiments, different shapes and configurations may be used. During deployment, the stent 100 expands. When the stent 100 expands, certain portions of the stent 100, such as Y-shaped sections 108a, b, undergo deformation so that the stent can change shape from its unexpanded to its expanded condition. During this expansion, other portions of the stent 100, such as straight section 106, do not undergo any substantial deformation, i.e., they undergo no or only minimal deformation.

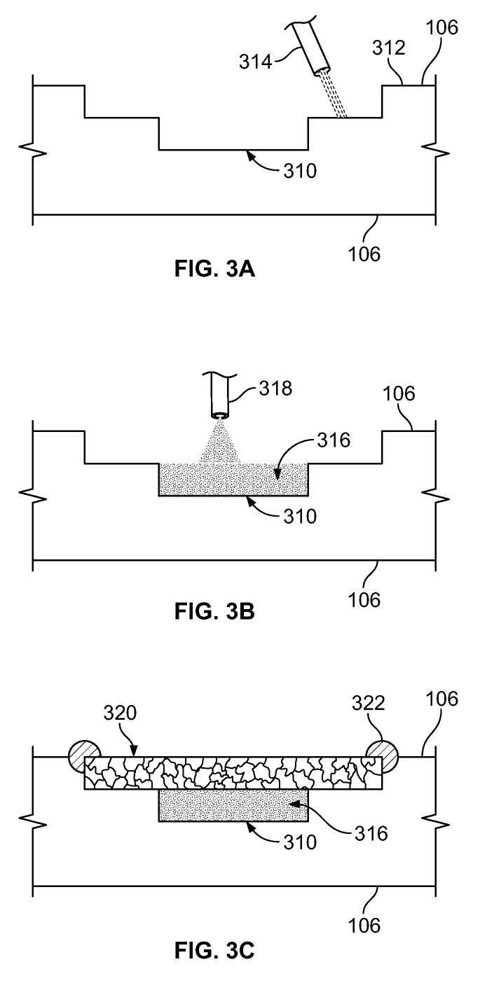

Turning to FIGS. 3a-c, a coating method that may be used to coat a medical device in accordance with certain embodiments of the present invention will be described. This method may include some or all of the steps identified in the figures. It may include other steps, as well as modifications to the identified steps. Although in FIGS. 3a-c, a portion of a stent is shown, any expandable medical device may be coated using the identified steps.

FIG. 3a shows a method step in which a reservoir 310 may be created within the straight section 106 of the stent strut. In the example, the reservoir 310 is created in an outer surface 312 of the straight section 106; however, other arrangements are possible. For instance, the reservoir 310 may be formed on an inner surface of the straight section 106. This step may be repeated to create as many reservoirs 310 as desired.

Turning back to FIG. 1, since adjacent straight sections, which include the reservoirs, are separated by Y-shaped sections, a plurality of disconnected reservoirs may be created on a target surface(s) of the stent.

Any suitable technique may be used for creating the reservoirs. For example, laser ablation, machining (e.g., drilling), and etching (e.g, wet/dry etching) may be used. In the example, a laser 314 is being used to ablate stent material and to create reservoir 310.

FIG. 3b illustrates a method step in which the reservoir 310 is being loaded with therapeutic agent 316. The stent may be loaded with therapeutic agent 316 by any suitable methods including, but not limited to, dipping, spraying, rolling, brushing, vapor deposition, and/or injection. In the example, the reservoir 310 is being spray coated with a spray nozzle 318.

FIG. 3c shows another method step that may be performed in accordance with certain embodiments of the present invention. In this figure, it can be seen that a porous coating 320 may be applied over the reservoir 310. Conventional coating techniques may be used for applying the porous coating 320. For example, conventional electrochemical deposition, electroplating, and physical vapor deposition techniques may be used for applying the porous coating 320.

The porous coating 320 may be secured to the straight section 106 by any suitable means. For example, as seen in FIG. 3c, the porous coating 320 may be laser welded to the straight section 106. These welds 322 may facilitate the securing of the porous coating 320 to the stent. In the example, it can also be seen that the surface area covered by the porous coating 320 is greater than the surface area covered by the reservoir 310. Using this arrangement, heat that is generated during the welding process can be dissipated, due to spacing from the reservoir 310, so as to avoid damaging the therapeutic agent 316 stored in the reservoir 310.

In vivo, therapeutic agent 316 stored in the reservoir 310 may release through pores in the porous coating 320 for delivery to target tissue of a patient. The porosity of the porous coating 320 may be used to contain and regulate the release of therapeutic agent 316 from the reservoir 310.

Any number of reservoirs may be used. Likewise, the size of each reservoir may be varied. Moreover, the types of therapeutic agent stored may be varied from reservoir to reservoir.

With respect to the porous coatings, the porosity of each coating may be different to vary the elution rate of the therapeutic agent from different reservoirs. Further, the concentration of reservoirs may vary from one section of the stent to another. Other arrangements may also be used to optimize drug delivery.

In these examples, since the reservoirs are located only on straight sections of the stent, which has limited or no deformation, damage to the coating(s) can be limited and/or prevented. By locating the reservoirs on or in the portion(s) of the stent that do not undergo any substantial deformation, more fragile stent coating(s), such as ceramic and other non-polymeric coatings, may be used while reducing the risk that these coatings could crack or flake, or otherwise become damaged during the stresses/strains that occur during stent expansion.

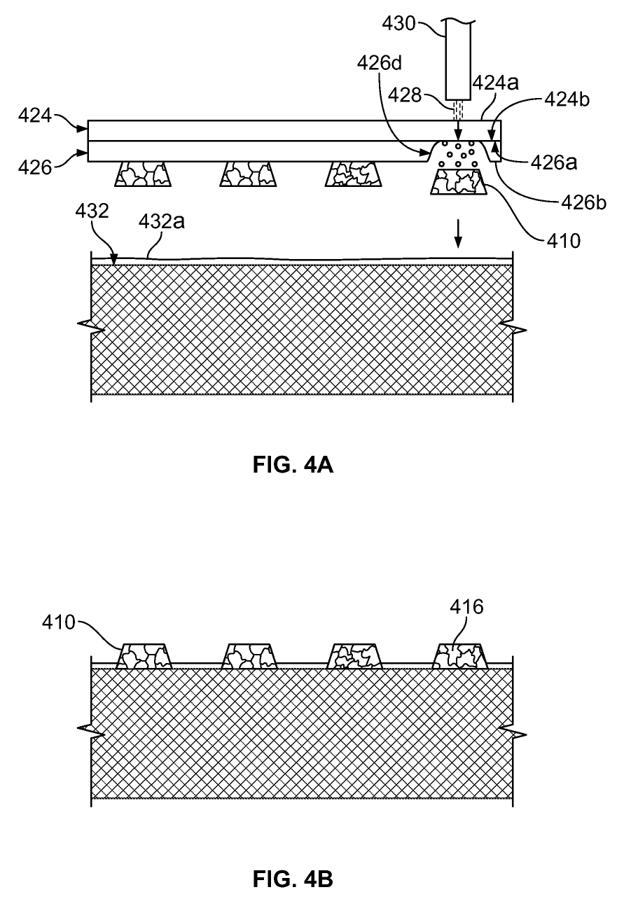

Turning to FIGS. 4a-b, a coating method that may be used to coat a medical device in accordance with another embodiment of the present invention will be described. This method may include some or all of the steps identified in the figures. It may include other steps, as well as modifications to the identified steps.

As shown in FIG. 4a this method may comprise the step of providing a medical device. As with the previous embodiments, although a stent 400 is shown in the figures, any medical device may be used.

The method may also include providing one or more reservoirs 410. The reservoirs 410 are intended to be loaded with therapeutic agent in such a manner as to hold the therapeutic agent until implantation of the medical device at which time the therapeutic agent elutes from the reservoirs 410. The reservoirs 410 may be porous for this purpose. The reservoirs 410 may be made of any suitable material, for instance, bio-degradable materials such as iron and magnesium may be used. Likewise, any suitable sizes and shapes may be used for the reservoirs 410. In the example, the reservoirs 410 are conically shaped; however, other shapes and sizes may be used. The reservoirs 410 shown in the example are merely illustrative and are not to scale. In use, the reservoirs 410 may be, for example, about 1 to 10 microns in height. The reservoirs may have any suitable porosity, such as in the a micro-porous and/or nano-porous range.

Any suitable techniques may be used for forming the reservoirs 410 including, but not limited to, metal printing and laser sintering. Metal printing and laser sintering will now be described in more detail.

Metal printing is used to produce three-dimensional objects from powdered materials. The metal printing process is described in detail in “Metal Printing Process Development of a New Manufacturing Process for Metal Parts,” written by C. van der Eijk, T. Mugaas, R. Karlsen, O. Asebo, O. Kolnes, and R. Skjevdal (October 2004), the entire contents of which are hereby incorporated by reference. See also “Introduction to the Metal Printing Process—Future Manufacturing Equipment for Advanced Materials and Complex Geometrical Shapes,” (October 2007), the entire contents of which are also hereby incorporated by reference.

With respect to laser sintering, this process is used to make objects from powder, such as by heating a material below its melting point until its particles adhere to one another. For example, selective laser sintering (SLS®, a registered trademark of 3D Systems, Inc.), is a manufacturing technique that uses a high powered laser (e.g., a carbon dioxide laser) to fuse small particles of plastic, metal, or ceramic powders into a mass representing a desired 3-dimensional object. The laser selectively fuses powdered material by scanning cross-sections generated from a 3-D digital description of the part (e.g. from a CAD file) on the surface of a powder bed. After each cross-section is scanned, the powder bed is lowered by one layer thickness, a new layer of material is applied on top, and the process is repeated until the part is completed. Laser sintering can be performed by machines called “Sinterstation SLS Systems,” which are manufactured by 3D Systems, Inc. Another manufacturer of selective laser sintering equipment is EOS GmbH of Munich, Germany.

In the example shown, the reservoirs 410 are loaded with therapeutic agent 416 prior to positioning of the reservoirs 410 onto a target surface 432 of the stent 400. In other examples, not shown, the reservoirs 410 may be loaded with therapeutic agent 416 following positioning of the reservoirs 410 onto a target surface of the stent 400. The reservoirs 410 may be loaded with therapeutic agent 416 by any suitable methods including, but not limited to, dipping, spraying, rolling, brushing, vapor deposition, and/or injection.

FIG. 4a illustrates components that may be used to conduct a laser-induced forward transfer method (LIFT), which will be described in more detail herein below. The LIFT method can use a laser 430 and a transfer member that comprises a translucent substrate 424 and a transfer material 426 which may be in the form of a film or foil.

In the example, an upper surface 426a of the transfer material 426 is positioned below a lower surface 424b of the translucent substrate 424. The transfer material 426 illustrated in the example is made from a titanium film or foil; however, other films and foils, including different biocompatible metals, may be used. In the example, the translucent substrate 424 is a sheet of glass; however, other suitable transparent and/or translucent materials may be used which allow a laser beam 428 to pass therethrough with sufficient intensity to vaporize a portion of the transfer material 426.

As is seen in FIG. 4a, once the reservoirs 410 are loaded with therapeutic agent 416, the reservoirs 410 may be positioned on a lower surface 426b of the transfer material 426. Adhesion of the reservoirs 410 to the transfer material 426 may be facilitated by an adhesive such as biocompatible glue or sugar.

In accordance with embodiments of the present invention, pick-and-place techniques, as are well known in the micro-systems and semi-conductor arts, may also be used to facilitate positioning of the reservoirs 410 as desired. Examples of pick-and-place techniques are described in “Micromanipulation and Micro-Assembly System,” authored by Cedric Clevy, Arnaud Hubert, and Nicolas Chaillet, the entire contents of which are hereby incorporated by reference.

Once the reservoirs 410 are properly positioned, the translucent substrate 424, transfer material 426, and reservoirs 410 may be arranged over the target surface 432 of the stent 400.

In accordance with embodiments of the present invention, to facilitate arrangement of the components, any X-Y-Z positioning system(s), as is well know in the art, may be used both before, after, and/or during the coating process. Likewise, conventional optical systems (e.g., CCD camera systems) may also be used to assist in positioning of components.

Turning again to FIG. 4a, now in a desired position, the LIFT method may be used to direct a laser beam(s) 428, at an upper surface 424a of the translucent substrate 424 using a laser 430 (e.g., an Nd-Yag Laser). It is contemplated by embodiments of the present invention that the laser beam may be pulsed.

The laser beam 428 can be focused so as to impinge upon the transfer material 426 through the translucent substrate 424. The focused laser beam 428 vaporizes a portion 426d of the transfer material 426, creating a pocket of trapped gases (within portion 426) between the translucent substrate 424 and the reservoir 410. The gas expands, e.g., within a fraction of a second, thereby ejecting one or more reservoirs 410 towards a target surface 432 of the stent 400. In the example, the outer surface of the stent is the target surface 432.

As is seen in FIG. 4a, the stent 400 and/or surfaces of the reservoirs 410 may be preconditioned to facilitate receipt of the reservoirs 410 on the target surface 432. For example, the target surface 432 may be etched using laser 430 and/or an additional laser (not shown). The etching process may be performed prior to and/or during the coating process. For example, the stent 400 may be rotated during coating so that one portion of the stent 400 may be etched with one laser while another portion may be coated using another laser.

In the example, the target surface 432 may be etched so that a thin layer (432a) of the target surface 432 melts. Once the reservoirs 410 are positioned on the target surface 432, the stent can cool, thus returning the target surface 432 to a solid state to secure the reservoirs 410 thereon. Still other surface activation methods may be used. Further, bio-compatible adhesives (e.g., glue, sugar, etc.) may also be used.

In certain embodiments of the present invention, the LIFT method may be performed in a vacuum or inert gas atmosphere (e.g., a vacuum chamber) to prevent damage to materials from exposure to oxygen and moisture.

As stated herein above, the LIFT method utilizes lasers to transfer materials. The LIFT method can be performed without solvents, which allows the deposition of layers without the need to identify suitable solvent systems or to obtain a polymer that can be evaporated thermally. The LIFT method can be used to transfer materials with a UV laser, by providing a release layer which absorbs the laser light at the irradiation wavelength and produces enough energy to vaporize the transfer material.

The LIFT method is described in detail in “Laser-Induced Forward Transfer (LIFT),” Paul Scherrer Institut, in collaboration with the Laboratory for Functional Polymers at EMPA, the entire contents of which are hereby incorporated by reference. See also “Laser Induced Forward Transfer: A New Approach for the Deposition of High T_{c} Superconducting Thin Films,” J. Mater Res., Vol. 4, No. 5, September/October 1989, E. Fograssy, C. Fuchs, F. Kerherve, G. Hauchecorne, and J. Perriere; “Nano-droplets Deposited in Microarrays by Femtosecond Ti:Sapphire Laser-Induced Forward Transfer,” David P. Banks, Christos Grivas, John D. Mills, and Robert Eason, Optoelectronics Research Centre, University of Southhampton, SO17 IBJ, United Kingdom, and Ioanna Zergioti, National Technical University of Athens, Physics Department, 15780 Zografu, Athens, Greece; “Excimer Laser Forward Transfer of Mammalian Cells Using a Novel Triazene Absorbing Layer,” Applied Surface Science 252 (2006) 4743-4747, A. Doraiswamy, R. J. Narayan, T. Lippert, L. Urech, A. Wokaun, M. Nagel, B. Hopp, M. Dinescu, R. Modi, R. C. Y. Auyeung, and D. B. Chrisey; “Preparation of Functional DNA Microarrays Through Laser-Induced Forward Transfer,” Applied Physics Letters, Volume 85, Number 9, August 2004, P. Serra, M. Colina, and J. M. Fernandez-Prada, L. Sevilla, and J. L. Morenza; “Femtosecond Laser-Induced Forward Transfer (LIFT): A Technique for Versatile Micro-Printing Applications;” and “Laser Induced Forward Transfer (LIFT) as a Microprinting Process,” University of Alberta Electrical and Computer Engineering website, Oct. 28, 2007, the entire contents of which are hereby incorporated by reference.

Turning to FIG. 4b, it can be seen that any numbers of reservoirs 410 may be applied to a target surface 432 of the stent 400. Thus, a plurality of discontinuous (e.g., stand alone), porous, and therapeutic agent loaded reservoirs may be formed. The porosities and types of therapeutic agent may vary from reservoir to reservoir. Likewise, the concentration of reservoirs may vary from one portion of the stent to another.

In these examples, since the reservoirs are discontinuous, i.e., not attached to each other, they do not impart stresses/strains on adjacent reservoirs as the stent is expanded. In this way, damage to the coating(s) can be limited and/or prevented. Thus, more fragile stent coating(s), such as ceramic and other non-polymeric coatings, may be used while reducing the risk that these coatings could crack or flake, or otherwise become damaged during the stresses/strains that occur during stent expansion.

While various embodiments have been described, other embodiments are possible. It should be understood that the foregoing descriptions of various examples of methods for coating medical devices are not intended to be limiting, and any number of modifications, combinations, and alternatives of the examples may be employed to facilitate drug delivery.

The term “therapeutic agent” as used herein includes one or more “therapeutic agents” or “drugs.” The terms “therapeutic agents” or “drugs” can be used interchangeably herein and include pharmaceutically active compounds, nucleic acids with and without carrier vectors such as lipids, compacting agents (such as histones), viruses (such as adenovirus, adenoassociated virus, retrovirus, lentivirus and -virus), polymers, hyaluronic acid, proteins, cells and the like, with or without targeting sequences.

Specific examples of therapeutic agents used in conjunction with the present application include, for example, pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; anti-proliferative agents such as, without limitation, enoxaprin, everolimus, zotarolimus, angiopeptin, rapamycin, angiopeptin, monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitrofurantoin; anesthetic agents such as lidocaine, bupivacaine, and ropivacaine; nitric oxide (NO) donors such as linsidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, structurelet receptor antagonists, anti-thrombin antibodies, anti-structurelet receptor antibodies, enoxaparin, hirudin, Warfarin sodium, Dicumarol, aspirin, prostaglandin inhibitors, structurelet inhibitors and tick antistructurelet factors; vascular cell growth promoters such as growth factors, growth factor receptor antagonists, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; survival genes which protect against cell death, such as anti-apoptotic Bcl-2 family factors and Akt kinase; and combinations thereof. Cells can be of human origin (autologous or allogenic) or from an animal source (xenogeneic), genetically engineered if desired to deliver proteins of interest at the insertion site. Any modifications are routinely made by one skilled in the art.

Polynucleotide sequences useful in practice of the application include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor α and β, structurelet-derived endothelial growth factor, structurelet-derived growth factor, tumor necrosis factor α, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMPs”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMPs are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNAs encoding them.

The examples described herein are merely illustrative, as numerous other embodiments may be implemented without departing from the spirit and scope of the exemplary embodiments of the present application. Moreover, while certain features of the application may be shown on only certain embodiments or configurations, these features may be exchanged, added, and removed from and between the various embodiments or configurations while remaining within the scope of the application. Likewise, methods described and disclosed may also be performed in various sequences, with some or all of the disclosed steps being performed in a different order than described while still remaining within the spirit and scope of the present application.

What is claimed is:

1. An expandable medical device comprising:
one or more first portions that do not undergo substantial deformation during expansion of the medical device; and
one or more second portions that undergo substantial deformation during expansion of the medical device;
one or more reservoirs in or on the first portions of the medical device;
therapeutic agent loaded in the reservoirs; and
a non-polymeric porous coating over each of the reservoirs loaded with therapeutic agent.

2. A medical device according to claim 1, wherein the porous coating comprises a material selected from the group consisting of metals, ceramics, and bio-ceramics.

3. A medical device according to claim 1, wherein the surface area of each porous coating is greater than the surface area of the reservoir it covers.

4. A medical device according to claim 1, wherein the porous coating is secured to the medical device by laser welding.

5. A medical device according to claim 1, wherein the reservoirs are created in an outer surface of the medical device.

6. A medical device according to claim 1, wherein the reservoirs are created using a technique selected from the group consisting of laser ablation, machining, and etching.

7. A medical device according to claim 1, wherein the medical device is a stent, the first portions are portions of the stent that do not undergo substantial deformation during expansion of the stent, and the second portions are portions of the stent that undergo substantial deformation during expansion of the stent.

8. A method for applying therapeutic agent to a medical device, the method comprising the steps of:
providing an expandable medical device having one or more first portions that do not undergo substantial deformation during expansion of the medical device and one or more second portions that undergo deformation during expansion of the medical device;
providing one or more reservoirs in or on the first portions of the medical device;
loading therapeutic agent loaded in the reservoirs; and
providing a non-polymeric porous coating over each of the reservoirs loaded with therapeutic agent.

9. A method according to claim 8, wherein the porous coating comprises a material selected from the group consisting of metals, ceramics, and bio-ceramics.

10. A method according to claim 8, wherein the surface area of each porous coating is greater than the surface area of the reservoir it covers.

11. A method according to claim 8, wherein the porous coating is secured to the medical device by laser welding.

12. A method according to claim 8, wherein the reservoirs are created in an outer surface of the medical device.

13. A method according to claim 8, wherein the reservoirs are created using a technique selected from the group consisting of laser ablation, machining, and etching.

14. A method according to claim 8, wherein the medical device is a stent, the first portions are portions of the stent that do not undergo substantial deformation during expansion of the stent, and the second portions are portions of the stent that undergo substantial deformation during expansion of the stent.

15. A method for applying therapeutic agent to a medical device, the method comprising the steps of:
providing a medical device;
providing a transfer member comprising a translucent substrate and a layer of transfer material;
attaching one or more reservoirs to the transfer material;
positioning the transfer member with the reservoirs adjacent the medical device, with the reservoirs facing the medical device; and
directing a laser beam at the transfer member such that the laser beam passes through the translucent substrate, thereby vaporizing a portion of the transfer material, creating a pocket of trapped gas between the translucent substrate and reservoir, and forcing the reservoir from the transfer member to the medical device.

16. A method according to claim 15, wherein the reservoirs are loaded with therapeutic agent.

17. A method according to claim 15, wherein the reservoirs are formed of a non-polymeric material.

18. A method according to claim 15, wherein the reservoirs are secured to the medical device using a laser.

19. A method according to claim 15, further comprising loading a first reservoir with a first therapeutic agent and a second reservoir with a second therapeutic agent that is different than the first therapeutic agent.

20. A method according to claim 15, wherein the medical device is a stent.
